(19) United States
(12) Patent Application Publication
(10) Pub. No.: US 2004/0247588 A1
(43) Pub. Date: Dec. 9, 2004

(54) Formulations of modified antibodies and methods of making the same

(76) Inventors: Robert E. Johnson, Chesterfield, MO (US); Lavinia M. Lewis, Chesterfield, MO (US); Hong Qi, San Diego, CA (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/634,199

(22) Filed: Aug. 5, 2003

Related U.S. Application Data
(60) Provisional application No. 60/406,412, filed on Aug. 28, 2002.

(51) Int. Cl. A61K 39/395

(52) U.S. Cl. 424/130.1

(57) ABSTRACT

The present invention is directed to novel formulations of modified antibodies and to methods of producing the same. The modified antibodies formulated according to the present invention each comprise an antibody fragment covalently attached to at least one nonproteinaceous polymer, such as poly(ethyleneglycol). CDP870 is an example of one such modified antibody, a therapeutic modified antibody. One method disclosed herein involves the removal from a solution of the modified antibody of molecules capable of adversely affecting the stability or solubility of the modified antibody after lyophilization (e.g., by dialysis or diafiltration), followed by lyophilization of the modified antibody. Another method involves concentration of a modified antibody solution by equilibrium dialysis. The methods of the present invention can be used to produce formulations suitable for use in subcutaneous and parenteral injection, including high concentration formulations.

### CROSS-REFERENCE TO RELATED APPLICATIONS

This application claims the benefit of U.S. Provisional Application Ser. No. 60/406,412, filed Aug. 28, 2002.

### FIELD OF THE INVENTION

The present invention relates to lyophilized and reconstituted lyophilized formulations of modified antibodies, including lyophilized forms of modified antibodies designed for use in producing high concentration formulations. The present invention also relates to high concentration formulations of modified antibodies formed using means other than lyophilization, such as by concentration equilibrium dialysis. The invention particularly relates to formulations of modified antibodies, preferably therapeutic modified antibodies, that include at least one nonproteinaceous polymer, such as poly(ethyleneglycol). One such therapeutic modified antibody is CDP870.

### BACKGROUND

Antibodies have been identified and developed for use in the diagnosis, prevention, and treatment of many different diseases and disorders. The utility of antibodies for use in such applications stems from their highly specific affinity for particular targets, such as target organisms, tissues, or even molecules. Examples of such antibodies discovered so far include the following. Antibodies with specificity for antigenic determinants of human tumor necrosis factor alpha (TNFα) have been identified, for use in the diagnosis, prevention, and treatment of various diseases associated therewith. Monoclonal antibodies against TNFα have been described in the literature. See, for example, murine monoclonal antibodies disclosed in, Meager et al., Hybridoma 6: 305-311 (1987); Fendly et al., Hybridoma 6: 359-370 (1987); Shimammoto et al., Immunology Letters 17: 311-318 (1988). Complementarity-determining region (“CDR”)-grafted antibodies specific for TNFα are disclosed in Rankin et al., British J. Rheumatology 34: 334-342 (1995). A humanized CDR-grafted modified antibody specific for TNFα, CDP870, is disclosed in international publication number WO 01/94585 A1.

Antibodies to TNFα are examples of particularly useful antibodies because TNFα as a pro-inflammatory cytokine that is released by and interacts with cells of the immune system. (WO 01/94585 A1, page 2). TNFα is released by macrophages that have been activated by lipopolysaccarides (LPS) of gram negative bacteria. (Id.) As such, TNFα is believed to be an endogenous mediator involved in the development and pathogenesis of endotoxic shock associated with bacterial sepsis. (Id.) TNFα has also been shown to be up-regulated in a number of human diseases, including chronic diseases such as rheumatoid arthritis, Crohn's disease, ulcerative colitis, and multiple sclerosis. (Id.) For patents disclosing antibodies to TNFα and their uses, see U.S. Pat. Nos. 6,248,471; 6,528,562; 5,654,407; 6,090,923; and 5,795,697, all of which are incorporated herein by reference. Antibodies to many other antigens implicated in other diseases and disorders are also known.

Antibodies are a type of protein. Like any protein, the biological activity of an antibody, such as its binding affinity, depends upon the conformational integrity of at least a core sequence of amino acids remaining intact while protecting the protein's multiple functional groups from degradation. The same principals regarding biological activity apply to antibody fragments, with a core sequence of amino acids comprising an antigen binding or variable region of a full-length antibody. Chemical and physical instability can each contribute to degradation of an antibody, antibody fragment, or other protein. Chemical instability can result from deamidation, racemization, hydrolysis, oxidation, beta elimination or disulfide exchange. Physical instability can result from denaturation, aggregation, precipitation, or adsorption, for example. The three most common protein degradation pathways are protein aggregation, deamidation, and oxidation. U.S. Pat. No. 6,267,958, col. 1, lines 29-40, citing Pikal, M. Biopharm. 3(9)26-30 (1990) and Arkawa et al. Pharm. Res. 8(3):285-291 (1991). See also, Stratton, Lewis et al. J. of Pharm. Sci. 90(12):2141-2148 (December 2001).

Modified antibodies include at least one moiety either attached directly to the antibody, or attached indirectly to the antibody through at least one linker. Antibodies are modified for a variety of different reasons, including but not limited to increase the stability of the antibody, to add a functional group to the antibody to be used to isolate the antibody, or to change the rate at which the antibody is eliminated from a subject after administration thereto. Partial degradation of a linker can result in rendering the moiety ineffective. Complete degradation of a linker results in detachment of a moiety from an antibody.

Antibodies modified by the covalent linkage of an antibody fragment to at least one nonproteinaceous polymer tend to have a considerably higher residence time in a subject, such that the antibody can have time to reach the biological target material and, if the antibody is a therapeutic antibody, to have a therapeutic effect on the target material. Nonproteinaceous polymers include, but are not limited to, poly(ethyleneglycol), poly(propyleneglycol), or poly(oxyalkylene) in the manner set forth in U.S. Pat. Nos. 4,640,835; 4,496,689; 4,301,144; 4,670,417; 4,791,192; all of which are incorporated by reference herein. Methods for attachment of poly(ethyleneglycol) polymer(s) to antibody fragments to make modified antibodies are disclosed in U.S. Pat. Nos. 6,113,906; 5,919,455; 5,643,575; and 5,932,462; and in EP 788 515, all of which are incorporated by reference herein.

CDP870, described in WO 01/94585 A1 (filed by Celltech R & D Ltd.), is a modified antibody, comprising an antibody fragment with a light chain and a heavy chain derived from a mouse monoclonal antibody having specificity for human TNFα, covalently linked to a succinimide moiety that is in turn covalently linked to a lysine residue covalently linked to two methoxypoly(ethyleneglycol) polymers (of approximately 20,000 Da each). (Id., page 9, line to page 10, line 2 and FIG. 13). The antibody sequences of CDP870 are vulnerable to degradation, as are any amino acid sequences. Additionally, the ring structure of the succinimide moiety, a component of the linker of each CDP870 molecule, has a tendency to open in the presence of a solution at high pH. Ishi, Yoshiharu, et al., Biophys. J. 50:75-80 (July 1986).

Lyophilization is a commonly employed technique for preserving proteins. Lyophilization, a freeze-drying procedure, is a process by which a material to be dried is first frozen, and the resulting ice or frozen solvent is removed by sublimation in the presence of a vacuum. An excipient can be included in a pre-lyophilized formulation to enhance stability of the material during the freeze-drying process and/or to improve the stability of the lyophilized product upon storage. U.S. Pat. No. 6,267,958, citing Pikal, M. Biopharm. 3(9)26-30 (1990) and Arkawa et al. Pharm. Res. 8(3):285-291 (1991).

In addition to being used to preserve proteins, lyophilization has been used to produce solutions of reconstituted proteins where the concentration of protein is different from that of the pre-lyophilized formulation used to produce the reconstituted proteins. U.S. Pat. No. 6,267,958 discloses one such use of lyophilization to produce a reconstituted formulation of protein that is about 2 to 40 times greater than the protein concentration in a mixture before lyophilization. (Col. 3, lines 19-22). The '958 patent discloses use of lyophilization to produce such concentrated reconstituted formulations of various types of antibodies, including monoclonal and polyclonal antibodies, humanized and human antibodies, and bispecific antibodies. The reconstituted antibody formulations disclosed in the '958 patent are stable isotonic reconstituted formulations, each comprising an antibody in amount of about 50 mg/ml to about 400 mg/ml and a diluent, wherein the reconstituted formulation has been prepared from a lyophilized mixture of the antibody and a lypoprotectant. (claim 1). The molar ratio of lypoprotectant to antibody in each reconstituted antibody formulation disclosed therein was about 100-510 mole lypoprotectant to one mole of antibody. (Id.)

Lyoprotectants disclosed in the '958 patent as being suitable for use in lyophilization of antibodies and other proteins according to the method disclosed therein include: “sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, sugar alcohols, e.g. glycerin, erythritol, glycerol, arabitol, zylitol, sorbitol, and mannitol; propylene glycol; polyethylene glycol; Pluronics; and combinations thereof.” ('958 patent, col. 9, lines 24-33). Sucrose and trehalose are described therein as being particularly preferred lyoprotectants. At no point does the '958 patent suggest that any non-proteinaceous polymer lyoprotectant, such as a poly(ethyleneglycol) polymer, could be used to stabilize an antibody or antibody fragment in solution or during lyophilization by covalent attachment thereto.

For another example of use of lyophilization to concentrate antibodies and other proteins, see international publication WO 97/04801 (filed by Genentech, Inc.). See, specifically, the disclosure of anti-IgE and anti-HER2 antibody formulations with a reconstituted concentration of between about 80 mg/ml to about 300 mg/ml, as disclosed on page 3 of the publication. This publication includes discussion of the use of lyoprotectants that is identical to that of the paragraph of the '958 patent, reproduced above.

Formulations of reconstituted lyophilized antibodies produced according to the methods disclosed in the '958 patent and in WO 97/04801 are sufficiently concentrated to be potentially suitable for use in injection, although, a more concentrated formulation would be desirable in order to decrease the volume required for administration. Unfortunately, the types of antibodies disclosed in the two references tend to be cleared rapidly from the body of a subject after injection, thus limiting the utility of such antibodies for most applications. Neither the '958 patent nor WO 97/04801 disclose antibodies modified with non-proteinaceous polymers, a type of modified antibody with considerably longer post-injection clearance rates than unmodified antibodies.

Liquid-based methods for concentrating proteins, such as ultrafiltration, ultracentrifugation, and equilibrium dialysis, enable one to avoid problematic reconstitution steps. However, such techniques have not previously been used to produce antibodies modified with nonproteinaceous polymers in a formulation with a concentration suitable for use in injection, much less highly concentrated formulations of CDP870.

It is an object of the present invention to provide a stable, concentrated, modified antibody formulation and a method of producing the same. It is a further object to provide a modified antibody solution in a concentration range sufficiently high to be useful in subcutaneous or parenteral injection.

### BRIEF SUMMARY OF THE INVENTION

Accordingly, the invention provides stable formulations of a modified antibody in a concentration range sufficiently high for use in subcutaneous or parenteral injection, including modified antibody formulations wherein the concentration of modified antibody is at least about 300 mg/ml, and methods of producing the same.

In one embodiment, the invention relates to a method of making a formulation of a modified antibody, comprising the steps of: (a) providing a pre-lyophilized modified antibody solution comprising the modified antibody and molecules capable of adversely affecting the stability or solubility of the modified antibody after lyophilization, wherein the modified antibody comprises an antibody fragment covalently attached to at least one nonproteinaceous polymer, (b) removing at least some of the molecules from the solution, and (c) lyophilizing the dialyzed solution, thereby producing a lyophilized modified antibody formulation. The molecules are preferably removed in step (b) by either dialysis or by diafiltration. The lyophilized modified antibody formulation can be reconstituted in a pharmaceutically acceptable diluent to produce a reconstituted formulation with a concentration of modified antibody sufficiently high to be suitable for administration to a subject, by injection or by intravenous means.

In another embodiment, the invention relates to a lyophilized modified antibody formulation or a reconstituted modified antibody formulation produced according to the method described immediately above.

In yet another embodiment, the invention relates to a high concentration formulation of a modified antibody, comprising a modified antibody in a diluent for a concentration of at least about 300 mg/ml of modified antibody. This formulation can be produced by reconstituting a lyophilized modified antibody formulation, produced as described above, in a small volume of the diluent. The high concentration formulation can also be produced by concentrating a modified antibody solution using equilibrium dialysis.

### BRIEF DESCRIPTION OF THE DRAWINGS

FIG. 1 is a plot of change in % acidic species in a reconstituted formulation of CDP870 stored over time at three different temperatures (40° C., 25° C., and 5° C., respectively), as described in Example 5, below.

FIG. 2 is a plot of change in % basic species over time, in the same reconstituted formulation of CDP870 stored under the same conditions as described for FIG. 1, above.

FIG. 3 is a plot of change in % aggregates over time in the same reconstituted formulation of CDP870 stored under the same conditions as described for FIG. 1, above.

### DETAILED DESCRIPTION OF THE INVENTION

The term “antibody” is used herein in the broadest sense and specifically covers, but is not limited to, monoclonal antibodies (including full length monoclonal antibodies), polyclonal antibodies, multispecific antibodies (e.g., bispecific antibodies), modified antibodies, and antibody fragments, so long as they exhibit the desired biological activity.

“Antibody fragments” comprise a portion of a full length antibody, generally the antigen binding or variable region thereof. Examples of antibody fragments include Fab, Fab′, F(ab′)₂, and Fv fragments, diabodies, linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, monoclonal antibodies may be made by the hybridoma method first described by Kohler et al., Nature 256:495 (1975). The “monoclonal antibodies” may also be isolated from phage antibody libraries using the techniques described in Clackson et al, Nature 352:624-628 (1991) and Marks et al., J. Mol. Biol. 222:581-597 (1991), for example.

The term “chimeric antibodies” (immunoglobulins), as used herein refers to monoclonal antibodies wherein a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad Sci. USA 81:6851-6855 (1984)).

The term “hypervariable region,” as used herein, refers to the amino acid residues of an antibody, which are responsible for antigen-binding. The hypervariable region comprises amino acid residues from a “complementary determining region” or “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop”(i.e. residues 26-32 (L1), 50-52 (L2) and 91-96 (L3) in the light chain variable domain and 26-32 (H1), 53-55 (H2) and 96-101 (H3) in the heavy chain variable domain; Chothia Lesk J. Mol. Biol. 196:901-917 (1987)).

“Humanized” forms of non-human (e.g., murine) antibodies are chimeric antibodies, which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. For further details, see Jones et al., Nature 321:522-525 (1986); Riechmann et al, Nature 332:323-329 (1988); and Presta, Curr. Op. Struct. Biol. 2:593-596 (1992).

The modified antibody of the formulations of the present invention comprises an antibody fragment covalently attached to at least one nonproteinaceous polymer. The antibody fragment can be any antibody fragment, preferably a therapeutic antibody fragment. The antibody fragment preferably comprises a heavy chain and a light chain, such as the heavy chain and light chain of the antibody fragment component of CDP870. CDP870 is a member of a class of therapeutic modified antibodies with a therapeutic antibody fragment containing at least one CDR having specificity for human TNFα. The light chain of CDP870 comprises an amino acid sequence identified by SEQ ID NO:1, while the heavy chain comprises an amino acid sequence identified by SEQ ID NO:2, which correspond to SEQ ID NO:113 and SEQ ID NO:115 of WO 01/94585, respectively. The modified antibody is preferably CDP870.

At least one nonproteinaceous polymer covalently attached to the antibody fragment of the modified antibody is preferably poly(ethyleneglycol), poly(propyleneglycol), poly(oxyalkylene), or a derivative thereof. The nonproteinaceous polymer is more preferably poly(ethyleneglycol) or a derivative thereof, such as a methoxypoly(ethyleneglycol) polymer. The at least one nonproteinaceous polymer is preferably one which increases the residence time of the modified antibody in a host subject, after administration thereto, compared to the residence time of an antibody without any nonproteinaceous polymer component. The modified antibody is preferably more stable than the antibody fragment without modification. Each nonproteinaceous polymer preferably has a molecular weight of about 5,000 to about 50,000 Daltons (Dal), more preferably about 10,000 to about 40,000 Dal, even more preferably about 15,000 to about 30,000 Dal, most preferably about 20,000 Dal. In a particularly preferred embodiment, two nonproteinaceous polymers are covalently attached to the antibody fragment, either directly, or through a linker.

The at least one nonproteinaceous polymer is preferably covalently attached to the antibody fragment through a linker. Any stable biologically compatible linker is suitable for use in connecting the antibody fragment to the at least one nonproteinaceous polymer. When the modified antibody is present in a solution, the linker is preferably stable in the solution. The linker preferably includes a succinimide moiety. In a particularly preferred embodiment, the at least one nonproteinaceous polymer is covalently linked to the antibody fragment through a linker comprising a succinimide moiety. In CDP870, two methoxypoly(ethyleneglycol) polymers are covalently attached to a lysine residue that is linked through a succinimide moiety linked to a cysteine residue of the antibody fragment.

The structure of CDP870 is illustrated in Figure I, below:

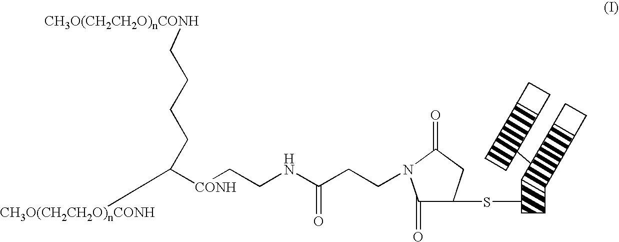

The C-terminal end of the heavy chain of the Fab fragment, shown on the right side of Figure I, includes a modified hinge region that is covalently linked to a succinimide moiety through a single thiol group attached thereto. A lysine residue is covalently linked to the succinimide moiety, and to each of the amines of the lysine residue is attached a methoxypoly(ethyleneglycol) polymer having a molecular weight of approximately 20,000 Da.

The antibody fragment of CDP870 comprises at least one CDR having specificity for human TNFα. The light chain of CDP870 comprises an amino acid sequence identified by SEQ ID NO:1, while the heavy chain comprises an amino acid sequence identified by SEQ ID NO:2. These two sequences correspond to SEQ ID NO:113 and SEQ ID NO:115 of WO 01/94585, respectively.

In the formulation of the present invention, the modified antibody is present in a solution wherein the concentration of modified antibody in the solution is suitable for injection, directly, or in the form of a more dilute formulation such as could be administered intravenously. The term “suitable for injection”, as used herein, refers to a solution that is not so viscous that it cannot be injected into a subject, using any means. The formulation of the present invention is preferably suitable for parenteral administration, for example, by injection using at least one commercially available injection means, including but not limited to, a hypodermic syringe, an autoinjector, a pen system, a dual chambered syringe, a needless syringe, or a microarray.

When the modified antibody is to be delivered in a more dilute form, the concentration of modified antibody in the formulation is preferably about 100 mg/ml and up to about 300 mg/ml, more preferably about 150 mg/ml to about 250 mg/ml, even more preferably about 190 mg/ml to about 210 mg/ml. When the modified antibody is to be delivered in a more concentrated form, the concentration of modified antibody in the formulation is preferably at least about 300 mg/ml, more preferably about 300 mg/ml to about 450 mg/ml, even more preferably about 310 mg/ml to about 440 mg/ml, most preferably over 400 mg/ml to about 440 mg/ml.

In another embodiment, the lyophilized modified antibody formulation of the present invention includes a buffer that maintains the pH of a reconstituted solution of the modified antibody at a lower pH where the reconstitution time for the lyophilized modified antibody is higher than it is at a higher pH. Preferred pH ranges for rapid reconstitution times are pH of 2.5 to about 6, more preferably a pH of about 4 to about 6, more preferably about 4.8 to about 6, even more preferably about 5 to about 6. The buffer is preferably a lactic acid buffer.

In yet another embodiment, the modified antibody formulation of the present invention includes a buffer configured to maintain the formulation at a pH at which the modified antibody is stable. When the modified antibody is CDP870, buffer of the reconstitution solution preferably maintains the pH of the resulting reconstituted modified antibody formulation at about pH 2.5 to about 6.0, more preferably at about pH 3.0 to about 5.5, even more preferably at about pH 4.0 to about 5.5. The preferred pH ranges given immediately above have been found to stabilize CDP870, particularly, minimizing the risk of opening the ring of the malemide residue of the CDP870 linker.

When a buffer is included in the modified antibody formulation of the present invention, it is preferably a nonvolatile organic buffer, such as a histidine, a lactic acid, or a succinate buffer. Volatile organic acid buffers can be used. However, non-volatile organic buffers are preferred because they are less likely to loose their buffering capacity or change pH over time. Use of lactic acid is particularly preferred because lyophilized formulations of modified antibodies containing a lactic acid buffer tend to have faster reconstitution times than other buffers.

The modified antibody formulation of the present invention preferably further comprises an excipient or a co-solvent, such as a polyol, or a combination of the excipient and the co-solvent. When lyophilization is used to produce the modified antibody formulation, an excipient, a co-solvent, or both an excipient and a co-solvent are preferably selected that promote reconstitution of the lyophilized form of the modified antibody to produce the reconstituted formulation of the present invention. Both the excipient and co-solvent preferably independently, or synergistically increase the stability of the modified antibody in the formulation, regardless of how the modified antibody formulation was produced. A description of excipients, co-solvents, and other suitable additional components of the formulations of the present invention is provided as part of the description of the methods of the present invention, herein below.

It is anticipated that any one of a number of different methods could be used to make the concentrated modified antibody formulation of the present invention. Two such methods are disclosed herein, at least one of which is a novel method not previously used to produce a modified antibody formulation, much less the formulations of the present invention. In one method, concentrated formulations of the present invention are made using a concentrating dialysis system, such as a Slide-A-Lyzer® Cassette and Concentrating Solution (Pierce Chemical Company). In the other method, dialysis is used in combination with lyophilization to produce higher or lower concentration formulations of a modified antibody. This last method is described in greater detail, below.

In embodiment of the method of the present invention, a pre-lyophilized formulation of the modified antibody described herein above is dialyzed and lyophilized. The pre-lyophilized formulation comprises molecules, small enough to be removed by dialysis, that are capable of adversely affecting the stability or solubility of the modified antibody after lyophilization. The molecules removed to produce the dialyzed formulation are all preferably smaller in size than the modified antibody. The molecules are even more preferably selected from the group consisting of salts or any other molecules likely to prevent the reconstitution of lyophilized modified antibody the volume of liquid required to produce a reconstituted modified antibody of any particular desired concentration. Dialysis is carried out until at least some of the molecules are removed. Preferably at least 40% of the molecules are removed in the dialyzing step, more preferably at least 60% of the molecules are removed, even more preferably at least 80% of the molecules are removed, even more preferably at least 90% of the molecules are removed. When the molecules comprise salts, at least 90%, more preferably at least 95%, even more preferably at least 98% of the salts present in the pre-lyophilized formulation are removed.

The method of the invention also preferably includes an exchange of buffers as part of the dialysis step, particularly when the buffer present in solution prior to the exchange is a buffer, such as an acetate buffer, that is at least partially volatile when lyophylized. The at least partially volatile buffer is preferably exchanged with a nonvolatile buffer, more preferably a nonvolatile organic buffer, such as succinate, citrate, ascorbate, histidine, maleate, or lactic acid. Alternatively, the at least partially volatile buffer is exchanged with an inorganic acid, such as hydrochloric acid.

In an alternative embodiment, the molecules present in the pre-lyophilized modified antibody solution capable of adversely affecting the stability or solubility of the modified antibody after lyophilization are salts, and both salt removal and buffer exchange are carried out by diafiltration. When diafiltration is used, the end point of salt removal can be determined by a conductimeter measurement.

The effectiveness of any such molecule removal or buffer exchange step, whether done by dialysis or diafiltration will depend upon a number of different factors, including the concentration of the molecules in the exchange buffer used, the number of times the exchange buffer is changed during dialysis or diafiltration, and the amount of time spent in the molecule removal or buffer exchange step.

The dialysis step can result in an increase in the volume of dialyzed modified antibody, compared to the volume of the pre-dialyzed solution. The more the volume the dialyzed solution increases, the more preferable it is to concentrate the modified antibody prior to the lyophilization step, in order to optimize the amount of antibody recovered in the lyophilization step. Any conventional means can be used to concentrate the modified antibody prior to dialysis, including, but not limited to, ultrafiltration, affinity purification, and diafiltration.

Once at least some of the molecules have been removed from the modified antibody solution by dialysis or other means described above, lyophilization is used to remove water from the resulting modified antibody solution. Any conventional means of lyophilization can be used to dialyze the modified antibody solution in the method of the present invention. The lyophilization cycle used in the present method can include any one of a variety of cycle times, and can include at least one annealing step. Drying temperatures and freezing methods can also vary in the lyophilization cycle used in the present method.

The lyophilized modified antibody can either be stored in lyophilized form, another product of the present invention, or reconstituted in a solution to make a high concentration formulation of modified antibody of the present invention, described above. The pre-lyophilized concentration of CDP870 can be up to about 200 mg/ml, more preferably up to about 100 mg/ml, even more preferably up to about 66.7 mg/ml.

At least one excipient is preferably included in the pre-lyophilized formulation to enhance stability of the lyophilized product upon storage. See Pikal, M. Biopharm. 3(9)26-30 (1990) and Arakawa et al. Pharm. Res. 8(3): 285-291. Any pharmaceutically acceptable excipient can be included in the pre-lyophilized formulation and remain in the lyophilized formulation produced therefrom. The excipient is preferably a lyoprotectant, a solubilizing agent, a surfactant, a bulking agent, a pharmaceutically acceptable preservative, or a combination or mixture of two or more of the above.

When the lyophilized modified antibody is to be stored in lyophilized form, it preferably further comprises a lypoprotectant, preferably a non-reducing sugar such as sucrose, mannitol, sorbitol or trehalose. In another embodiment, the lyophilized modified antibody further comprises an amino acid, such as histidine or arginine, preferably in the form of a buffer.

In another embodiment, the lyophilized modified antibody further comprises a solubilizing agent, such as a cyclodextrin. Suitable cyclodextrins for use in the lyophilized modified antibody include, but are not limited to hydroxypropyl β-cyclodextrin and sulfobutylether β-cyclodextrin.

In yet another embodiment, the lyophilized modified antibody further comprises a surfactant, preferably a nonionic surfactant. Nonionic surfactants suitable for use in the lyophilized formulations of the present invention include, but are not limited to, polysorbates (e.g. polysorbates 20 or 80); poloxamers (e.g. poloxamer 188); sorbitan esters and derivatives, such as polyoxyethelenesorbitan monolaurates (e.g., TWEEN® 20 or TWEEN® 80, Uniqema, a business unit of ICI Americas Inc., New Castle, Del., USA); Triton; sodium dodecyl sulfate (SDS); sodium laruel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), poly(ethyleneglycol), poly(propylglycol), and copolymers of ethylene and propylene glycol (e.g., Pluronics, PF68 etc.). The surfactant is more preferably TWEEN® 20 or TWEEN® 80, or a mixture of TWEEN® 20 and 80. The surfactant serves as a wetting agent, and preferably aids in reconstitution of the lyophilized modified antibody. The surfactant is preferably present in the pre-lyophilized formulation in an amount from about 0.001% to about 0.5%, more preferably from about 0.005% to about 0.05%, most preferably about 0.01%

In other embodiments of the invention, the lyophilized modified antibody further comprises a bulking agent, such as manitol or glycine. The bulking agent preferably allows for the production of a uniform lyophilized cake, without excessive pockets therein.

In another embodiment of the method of the present invention, the lyophilized modified antibody is reconstituted in the presence of a pharmaceutically acceptable preservative. The preservative can be present in the lyophilized modified antibody or in the recontstitution solution. Suitable preservatives non-restrictively include mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and ascorbic acid and salts thereof.

In an alternative embodiment, the reconstitution solution further comprises a buffer, such as the buffer described in the description of an embodiment of the modified antibody formulation of the present invention, herein above.

The present invention is also directed to a method of treatment or prophylaxis of a disease, comprising providing the modified antibody formulation of the invention, described herein above, and delivering a pharmaceutically effective dose of the formulation to a subject to treat or prevent a disease associated with a disease antigen. The disease antigen is preferably TNFα. The disease is preferably one associated with TNFα, such as, but not limited to: primary biliary cirrhosis; Myelodysplastic syndrome; chronic variable immunodefficientcy; treatment refractory sarcoidosis; diffuse lung disease, such as pulmonary fibrosis that is idiopathic or secondary to RA, or acute interstitial pneumonitis; vasculitis, such as Wegeners vasculitis, polyarteritis nodosa, temporal arteritis, IgA nephropathy (Henoch-Schonlein Purpura); crescentic nephritisjuvenile treatment resistant uveitis; adult treatment resistant uveitis; primary sclerosing cholangitis, alcohol induced hepatitis, ulcerative colitis, inflammatory skin diseases, such as bullous pemhigoid, psoriasis, and pemphigus vulgaris; polyositis (dermatomyositis); or an inflammatory disease, such as endotoxic shock associated with bacterial sepsis or a chronic disease such as rheumatoid arthritis, Crohn's disease, ulcerative colitis, and multiple sclerosis. The disease treated according to the present method is, even more preferably, rheumatoid arthritis. The subject is preferably a mammal, more preferably a human being. When the disease antigen is TNFα and the subject is a human being, the modified antibody formulation is preferably a CDP870 formulation.

The present invention is further illustrated by the following examples. These examples are intended to be illustrative of the invention and should not be used to limit or restrict its scope.

### EXAMPLES

The following examples illustrate one or more of the embodiments of the invention described above. Note that all concentrations of high concentration formulations of CDP870 are reported in units of mg/g, because the solutions were viscous and the density of the solutions had not been determined at the time the measurements were made. Logically, the concentrations expressed in “mg/g” could be numerically much higher in terms of “mg/ml”. For reasons given above, we did not measure the density of any formulation having a CDP870 concentration of higher than 200 mg/ml in any of the Examples, below. Except where indicated below, solutions experiments described below were conducted at about 25° C., and at atmospheric pressure.

### Example 1

### Production of Concentrated CDP870 using Equilibrium Dialysis

A solution of about 200 mg/ml CDP870, 50 mM acetate buffer, and 125 mM NaCl was dialyzed using a Slide-A-Lyser® Dialysis Cassette (Pierce Chemical, Product #66451, a cassette with about a 10,000 MW cut off) and a Dialysate (5 mM succinate buffer, pH 5, prepared from succinic acid with pH adjusted with sodium hydroxide), as follows. Note that the dialysate could be any buffer.

1. A volume of 3 to 15 ml of 200 mg/ml CDP-870 required for an appropriate amount of active pharmaceutical ingredient (“API”) was injected into one of the ports of a dialysis cassette, using a syringe and an 18 gauge needle.

2. With the syringe needle still inside the cassette, an equal volume of air was withdrawn as the amount of sample injected into the cassette.

3. The cassette was placed in a large volume of dialysate, and stored in a cold room, at about 4° C.

3. The cassette was attached to a buoy in order to ensure that the dialysis cassette remained afloat in the dialysate. Dialysis was carried out under constant stirring, using a stir bar, at about 4° C., for 24 hours, with a maximum of four buffer exchanges at periodic intervals of three hours each.

4. Samples of the dialyzed CDP870 were withdrawn after injecting a volume of air equal to the volume of each sample to be withdrawn into one port, and withdrawing the sample from the opposite port.

After dialysis, the sample volume expanded, resulting in dilution of the CDP870 in the dialysis cassette. A four to five fold dilution effect was observed. The dialyzed CDP870 was concentrated prior to lyophilization, using either of two methods illustrated in Examples 2 or 3, below.

### Example 2

### Concentration of Dialyzed CDP870 using Centrifugation

A sample of the dialyzed CDP870 solution produced as described in Example 1, above, was concentrated by centrifugation in a Millipore ultrafree centrifugal filter unit (MW cut off of about 30,000; UFV4BTK25) at 5500 rpm at 5° C. for about 24 hours. The resulting solutions of CDP870 had concentrations of about 200 mg/ml to about 270 mg/g. The concentration in mg/ml could be higher

### Example 3

### Lyophilization to Produce a High Concentration Formulation of CDP870

Samples of dialyzed concentrated CDP870 produced as described in Example 2, above, were further concentrated by lyophilization followed by reconstitution in a small volume of an aqueous solution. Lyophilization was carried out using the following lyophilization cycle: freezing to −50° C., primary drying at −25° C., and secondary drying at 10° C., with a total cycle time of about 44 hours. Each sample contained 100 mg CDP870 prior to lyophilization. Pre- and post-lyophilization solution conditions were varied in order to identify solution conditions that could be used to produce high concentration formulations of CDP870. Solution conditions used pre and post-lyophilization, and the concentration of each resulting resuspension solution is set forth Table 1, below. Solution conditions were altered between and within each set of samples, in an attempt to discover pre- and post-lyophilization conditions capable of producing high concentration formulations of reconstituted CDP870.

TABLE 1

| | Pre-Lyophilization | | Concentration | Concentration |
|---|---|---|---|---|
| | Composition (100 mg/ml | Reconstitution | Determined | Estimated in |
| Sample | CDP870) | Solution Used | in mg/g | mg/ml |
| 1A | 0.5% sucrose, 0.01% | 100 mM acetate | 228 | 239 |
| | Tween 20, 10 mM | (pH 3.0), 0.01% | | |
| | succinate | Tween 20 | | |
| 1B | 0.5% mannitol, 0.01% | 100 mM acetate | 223 | 234 |
| | Tween 20, 10 mM | (pH 3.0), 0.01% | | |
| | succinate | Tween 20 | | |
| 1C | 0.5% HPCD, 0.01% | 100 mM acetate | 243 | 256 |
| | Tween 20, 10 mM | (pH 3.0), 0.01% | | |
| | succinate | Tween 20 | | |
| 1D | 3% mannitol, 0.01% | 100 mM acetate | 206 | 217 |
| | Tween 20, 10 mM | (pH 3.0), 0.01% | | |
| | succinate | Tween 20 | | |
| 1E | 3% HPCD, 0.01% | 100 mM acetate | 210 | 220 |
| | Tween 20, 10 mM | (pH 3.0), 0.01% | | |
| | succinate | Tween 20 | | |
| 2A | 3% sucrose, 0.0125% | WFI | 261 | 274 |
| | Tween 20, 5 mM succinate | | | |
| | (pH 4) | | | |
| 2B | 3% sucrose, 0.0025% | WFI | 273 | 287 |
| | Tween 20, 5 mM succinate | | | |
| | (pH 4) | | | |
| 2C | 10% sucrose, 0.0125% | WFI | 222 | 234 |
| | Tween 20, 5 mM succinate | | | |
| | (pH 4) | | | |
| 2D | 10% sucrose, 0.0025% | WFI | 216 | 226 |
| | Tween 20, 5 mM succinate | | | |
| | (pH 4) | | | |
| 2E | 3% sucrose, 0.0125% | WFI | 254 | 266 |
| | Tween 20, 5 mM succinate | | | |
| | (pH 5) | | | |
| 2F | 3% sucrose, 0.0025% | WFI | 251 | 264 |
| | Tween 20, 5 mM succinate | | | |
| | (pH 5) | | | |
| 2G | 10% sucrose, 0.0125% | WFI | 211 | 222 |
| | Tween 20, 5 mM succinate | | | |
| | (pH 5) | | | |
| 2H | 10% sucrose, 0.0025% | WFI | 207 | 218 |
| | Tween 20, 5 mM succinate | | | |
| | (pH 5) | | | |
| 3A | 1% sucrose, 0.0125% | WFI | 368 | 386 |
| | Tween 20, 5 mM succinate | | | |
| | (pH 5) | | | |
| 3B | 1% sucrose, 0.0125% | WFI | 336 | 353 |
| | Tween 20, 5 mM succinate | | | |
| | (pH 5) | | | |
| 3C | 1% sucrose, 0.0125% | WFI | 337 | 354 |
| | Tween 20, 5 mM succinate | | | |
| | (pH 5) | | | |
| 4A | 1% sucrose, 0.0125% | WFI | 341 | 358 |
| | Tween 20, 5 mM succinate | | | |
| | (pH 3.3) | | | |
| 4B | 1% sucrose, 0.0125% | WFI | 361 | 379 |
| | Tween 20, 5 mM succinate | | | |
| | (pH 3.3) | | | |
| 4C | 1% sucrose, 0.0125% | WFI | 330 | 347 |
| | Tween 20, 5 mM lactate | | | |
| | (pH 3.1) | | | |
| 4D | 1% sucrose, 0.0125% | WFI | 368 | 386 |
| | Tween 20, 5 mM lactate | | | |
| | (pH 3.1) | | | |
| 4E | 1% sucrose, 0.0125% | WFI | 310 | 325 |
| | Tween 20, 5 mM lactate | | | |
| | (pH 3.1) | | | |
| 5A | 1% sucrose, 0.0125% | WFI | 372 | 391 |
| | Tween 20, 5 mM lactate | | | |
| | (pH 3.1) | | | |
| 5B | 1% sucrose, 0.0125% | WFI | 351 | 369 |
| | Tween 20, 5 mM lactate | | | |
| | (pH 3.0) | | | |
| 5C | 1% sucrose, 0.0125% | WFI | 348 | 366 |
| | Tween 20, 5 mM lactate | | | |
| | (pH 3.0) | | | |
| 5D | 1% sucrose, 0.0125% | WFI | 301 | 316 |
| | Tween 20, 5 mM lactate | | | |
| | (pH 4.0) | | | |
| 5E | 1% sucrose, 0.0125% | WFI | 300 | 315 |
| | Tween 20, 5 mM lactate, | | | |
| | 5 mM lactate (pH 4.0) | | | |
| 5F | 1% sucrose, 0.0125% | WFI | 335 | 352 |
| | Tween 20, 5 mM citrate | | | |
| | (pH 3.0) | | | |
| 6A | 1% sucrose, 0.005% | 5% ethanol | 378 | 397 |
| | Tween 20, 5 mM lactate | | | |
| | (pH 3.0) | | | |
| 6B | 0% lyoprotectant, 0.005% | WFI | 399 | 419 |
| | Tween 20, 5 mM lactate | | | |
| | (pH 3.0) | | | |
| 6C | 3% sucrose, 0.005% | WFI | 392 | 411 |
| | Tween 20, 5 mM lactate | | | |
| | (pH 3.0) | | | |
| 6D | 0.5% sucrose + 1% | WFI | 399 | 419 |
| | glycine, 0.005% Tween | | | |
| | 20, 5 mM lactate (pH 3.0) | | | |
| 6E | 1% sucrose, 0.005% | 10% ethanol | 377 | 396 |
| | Tween 20, pH adjusted | | | |
| | with HCl (pH 3.0) | | | |
| 6F | 0% lyoprotectant, 0.005% | WFI | 374 | 392 |
| | Tween 20, pH adjusted | | | |
| | with HCl (pH 3.0) | | | |
| 6G | 3% sucrose, 0.005% | 10% ethanol | 356 | 374 |
| | Tween 20, pH adjusted | | | |
| | with HCl (pH 3.0) | | | |
| 6H | 1% sucrose, 0.005% | 0.001N HCl | 377 | 395 |
| | Tween 20, pH adjusted | | | |
| | with HCl (pH 3.0) | | | |
| 6I | 1% sucrose, 0.005% | WFI @37° C. | 388 | 408 |
| | Tween 20, no pH | | | |
| | adjustment (pH 5.0) | | | |
| 7A | 0.5% sucrose and 1% | WFI | 382 | 401 |
| | glycine, 0.005% Tween | | | |
| | 20, 5 mM lactate (pH 3.0) | | | |
| 7B | 0.5% sucrose and 1% | 0.1% phytic | 377 | 396 |
| | glycine, 0.005% Tween | acid | | |
| | 20, 5 mM lactate (pH 3.0) | | | |
| 7C | 0.5% sucrose and 1% | 0.1% phytic | 384 | 403 |
| | glycine, 0.005% Tween | acid @37° C. | | |
| | 20, 5 mM lactate (pH 3.0) | | | |
| 7D | 0.5% sucrose and 0.5% | WFI | 374 | 393 |
| | glycine and 0.3% HPCD, | | | |
| | 0.005% Tween 20, 5 mM | | | |
| | lactate (pH 3.0) | | | |
| 7E | 0.5% sucrose and 0.5% | 0.01N HCl | 393 | 413 |
| | glycine and 0.3% HPCD, | | | |
| | 0.005% Tween 20, 5 mM | | | |
| | lactate (pH 3.0) | | | |
| 7F | 0.5% sucrose and 0.5% | WFI @ 37° C. | 379 | 398 |
| | glycine and 0.3% HPCD, | | | |
| | 0.005% Tween 20, 5 mM | | | |
| | lactate (pH 3.0) | | | |

As shown in Table 1, above, it was found the most concentrated formulations of CDP870 could be obtained from a pre-lyophylization formulation of 100 mg/ml CDP870 in 0 to 3% sucrose, with or without 1% glycine or 1% glycine and 0.3% HPCD, 0.005% Tween 20, and either with a lactate buffer or with pH adjusted with HCl to pH 3.0. The resulting reconstituted solutions had a concentration ranging from 390 mg/ml to 419 mg/ml CDP870.

The high concentration reconstituted lyophilized dialyzed solutions were very viscous and dense. The concentration of each sample was determined in terms of milligrams per gram (mg/g), and converted to concentration units of milligrams per milliliter (mg/ml) by assuming the density of each solution to be about 1.05 g/ml.

The high concentrations obtained in this Example are thought to be due, in part to the inclusion of a dialysis step prior to lyophilization. When CDP870 was lyophilized without any dialysis step, the highest concentration of redissolved CDP80 obtained was only about 32 mg/ml. It was only after the incorporation of a dialysis step, whereby, unfavorable factors were removed and replaced with factors favorable to the promotion of dissolution of lyophilized material that we were able to obtain the high CDP870 concentrations illustrated in Table 1, above.

It was also found that the more time one spent reconstituting the lyophilized CDP870, the higher concentrations one could obtain. We found that higher concentrations could be obtained in less reconstitution time when a lactic acid buffer, at a pH of about 3 to about 5 was included in the solution of CDP870 prior to lyophilization. We also found that higher concentrations could be obtained when a pre-lyophylization concentration of CDP870 of less than 100 mg/ml was used, and when the pre-lyo CDP870 solution was stoppered under a vacuum prior to lyophilization (results not shown above).

Identity and structural integrity of the CDP870 samples concentrated by a combination of dialysis and lyophilization, as described above, was confirmed by ultraviolet absorption analysis, by isoelectric focusing, by SDS-PAGE, by CD and by SEC-HPLC. Usually, at modified antibody concentrations over 300 mg/ml, one would expect to run into protein stability problems, such as aggregation. However, we observed less than 5% aggregation in such samples, and the formulation did not appear to change in protein characteristics, in comparison to the control.

### Example 4

### Concentration using Equilibrium Dialysis

Samples of CDP870 were concentrated using the same type of dialysis apparatus and procedure set forth in Example 1, above, except that a Slide-A-Lyzer® Concentrating Solution (Pierce Chemical Company, Product# 66527; see “Instructions: Slide-A-Lyzer® Concentrating Solution” (August 1996; Pierce) is used as the dialysate, instead of a buffer described in Example 1, above. The Concentrating Solution is designed to reduce the volume of a solution contained within a Slide-A-Lyzer® Cassette.

The concentration of each of the resulting samples was determined, as shown in Table 2, below. As with the samples tested in Example 3, above, the samples below were assumed to have a density of 1.05 g/ml, and that figure was used to determine concentration in mg/ml from concentration in mg/g.

TABLE 2

| | | Concentration | Concentration |
|---|---|---|---|
| Sample | | Determined | Estimated in |
| Number | Pre-Dialysis Composition | in mg/g | mg/ml |
| 8A | 200 mg/ml CDP870, 125 mM | 295 | 310 |
| | NaCl, 50 mM acetate (pH 5.5) | | |
| 8B | 30 mg/ml CDP870, 10 mM | 248 | 260 |
| | succinate (pH 3.2) | | |
| 8C | 200 mg/ml CDP870, 125 mM | 315 | 330 |
| | NaCl, 50 mM acetate (pH 5.5) | | |
| 8D | CDP870 recycled from | 286 | 300 |
| | various experiments | | |

None of the CDP870 formulations produced by dialysis in the presence of the Concentrating Solution, as described above, were nearly as concentrated as the most concentrated of the dialyzed redissolved CDP870 formulations produced as described in Example 3, above. Nonetheless, present formulations were all over 250 mg/ml, within a concentration range that makes them useful for injection into mammalian subjects

### Example 5

### Stability Studies of Lyophilized and Reconstituted CDP870 Formulations

A solid lyophilized cake of CDP870 was produced by lyophilizing a concentrated dialyzed formulation of CDP870, as described in Examples 1 and 3, above, using specific conditions described herein below. Dialysis was carried out under conditions designed to promote desalting and exchange of a volatile acetate buffer for a nonvolatile buffer. Excipients were then added to the dialyzed CDP870 formulation to further promote reconstitution of the formulation after lyophilization. The composition of the formulation immediately prior to lyophilization is given in Table 3, below.

TABLE 3

| Component | Concentration or Volume |
|---|---|
| CDP870 | 66.7 mg/ml |
| Lactic Acid | 0.3 mg/ml |
| Sucrose | 33 mg/ml |
| Sodium Hydroxide | qs for a pH between 3.0 and |
| | 6.0 |
| Tween 20 | 0.03 mg/ml |
| WFI | qs |
| Fill Volume | 4.2 ml. |

Samples of the CDP870 solution described immediately above were lyophilized using the lyophilization cycle set forth in Table 5, below:

TABLE 5

| Step # | Steps | Description | Temp | Pressure | Duration |
|---|---|---|---|---|---|
| 1 | Loading | Hold | 25 | | 0:10 |
| 2 | Freezing | Ramp (T) | −40 | | 1:00 |
| 3 | Freezing | Hold | −40 | | 2:00 |
| 4 | Freezing | Ramp (T) | −21 | | 0:25 |
| 5 | Freezing | Hold | −21 | | 1:00 |
| 6 | Freezing | Ramp (T) | −40 | | 0:25 |
| 7 | Freezing | Hold | −40 | | 1:00 |
| 8 | Primary drying | Ramp (P) | −40 | 100 | 0:30 |
| 9 | Primary drying | Ramp (T) | −5 | 100 | 0:47 |
| 10 | Primary drying | Hold | −5 | 100 | 20:00 |
| 11 | Primary drying | Hold | −5 | 100 | 18:00 |
| 12 | Primary drying | Hold | −5 | 100 | 2:00 |
| 13 | Secondary | Ramp (T) | 10 | 100 | 0:20 |
| | drying | | | | |
| 14 | Secondary | Hold | 10 | 100 | 5:00 |
| | drying | | | | |
| 15 | Holding | Ramp (T) | 5 | 100 | 0:20 |
| 16 | Holding | Hold | 5 | 100 | 0:01 |
| 17 | Holding | | 5 | 100 | 0:01 |
| 18 | Holding | | 5 | 100 | 0:01 |
| 19 | Holding | | 5 | 100 | 0:01 |
| 20 | Holding | | 5 | 100 | 0:01 |
| 21 | Holding | | 5 | 100 | 0:01 |
| 22 | Ending values | | 5 | 100 | 0:01 |
| 23 | Pre-aeration | Ramp (P) | 5 | 0.1-0.5 psia | |
| 24 | Stoppering | | 5 | 0.1-0.5 psia | |
| 25 | Holding | | 5 | 0.1-0.5 psia | |
| 26 | Release the | Ramp (P, T) | 5-RT | 1 atm | |
| | vacuum | | | | |
| | manually | | | | |
| 27 | Unload | | 5- | 1 atm | |
| | | | RT | | |
| 28 | Abort the cycle | | | | |

The resulting lyophilized formulation was reconstituted in an aqueous solution for a CDP870 concentration of 200 mg/ml. The composition of the reconstituted formulation produced as described above is set forth in Table 6, below:

TABLE 6

| Formulation Component | Concentration or Volume |
|---|---|
| CDP870 | 200 mg/ml |
| Lactic Acid | 0.9 mg/ml |
| Sucrose | 100 mg/ml. |
| Sodium Hydroxide | qs for pH of 5.3 |
| Tween 20 | 0.1 mg/ml |
| WFI qs | 1 ml |

A set of stability tests, including appearance, SEC (sizing-measures aggregates), CEX (acidic or basic species), concentration were carried out on the CDP870 formulations prepared as described above. Based on the results of the stability study, it was projected that the CDP870 formulation would continue to remain stable for at least two years. Results of stability tests conducted on the reconstituted formulation are described in greater detail, below.

Samples of the CDP870 reconstituted formulation were stored at 5° C., 25° C., and 40° C. for 52 weeks, and stability tests were conducted at various time points. The CDP870 formulation was found to be stable throughout the period tested, with respect to formation of acidic species, basic species, aggregates, and depegylation. Specifically, CDP870 after reconstitution was found to have retained its stability and identity throughout the testing period. Projection of results from the real time data collected as described above indicate the formulation would be stable at both of the two temperatures tested (i.e. at 5° C. and at 25° C.) for at least one year. Results of the stability study are shown in Table 7, below:

TABLE 7

| Temperature | Time | change in | change in | change in | change in |
|---|---|---|---|---|---|
| (° C.) | (weeks) | % acidic species | % basic species | % aggregates | % depegylation |
| | 0 | 0 | 0 | 0 | 0 |
| 5 | 4 | 0.4 | 0 | 0.1 | 0 |
| 5 | 8 | 0.1 | 0 | 0 | 0 |
| 5 | 16 | 0.7 | 0 | 0.1 | 0 |
| 5 | 26 | 0.6 | 0 | 0 | 0 |
| 5 | 39 | 0 | 0.1 | 0.1 | 0 |
| 5 | 52 | 0.3 | 0.2 | 0 | 0 |
| | 0 | 0 | 0 | 0 | 0 |
| 25 | 1 | 0.1 | 0 | 0 | 0 |
| 25 | 2 | 0.4 | 0 | 0 | 0 |
| 25 | 4 | 0.4 | 0 | 0.2 | 0 |
| 25 | 8 | 0.3 | 0 | 0.2 | 0 |
| 25 | 12 | 0.3 | 0 | 0.1 | 0 |
| 25 | 16 | 0.9 | 0.1 | 0.3 | 0 |
| 25 | 26 | 1.2 | 0.1 | 0 | 0 |
| 25 | 39 | 0.4 | 0.3 | 0.5 | 0 |
| 25 | 52 | 1.3 | 0.5 | 0.7 | 0 |
| | 0 | 0 | 0 | 0 | 0 |
| 40 | 1 | 0.5 | 0 | 0.2 | 0 |
| 40 | 2 | 0.7 | 0 | 0.3 | 0 |
| 40 | 4 | 0.7 | 0.1 | 0.6 | 0 |
| 40 | 8 | 0.9 | 0.4 | 1 | 0 |
| 40 | 12 | 1.5 | 0.8 | 1.2 | 0.1 |
| 40 | 16 | 2.4 | 0.5 | 1.5 | 0.1 |
| 40 | 39 | 4.2 | 1 | 2.9 | 0.1 |

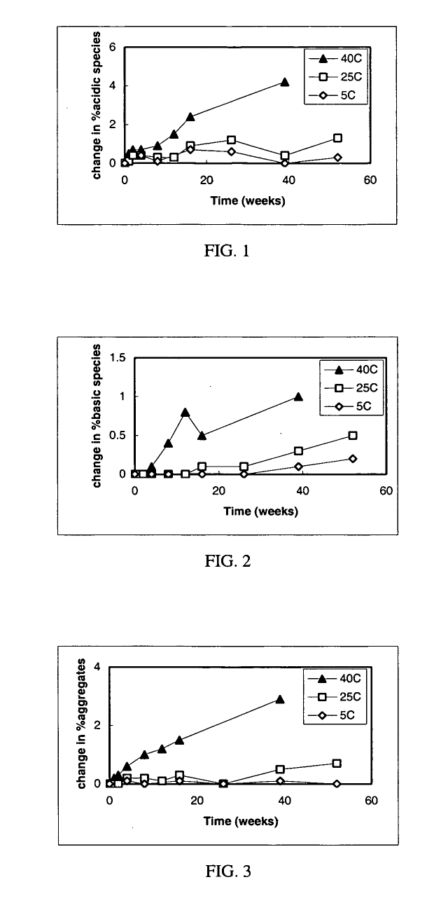

Stability results from Table 7, above, are shown in graphic format in FIGS. 1-3, as follows. FIG. 1 is a plot of the change in % acidic species observed in the formulation samples stored at each of the three temperatures studied, over time. FIG. 2 is a plot of the change in % basic species observed in the formulation samples stored at each of the three temperatures studied, over time. Finally, FIG. 3 is a plot of the change in % aggregates observed in the formulation samples stored at each of the three temperatures studied, over time. In all three figures, the samples stored at 5° C. were the most stable, showing the least increase in acidic species, basic species, or aggregation of all the samples tested. However, the samples stored at 25° C. were also very stable, and only showed slight increases in acidic species, basic species, or aggregation over time. Samples stored at 40° C. were less stable than those stored at lower temperatures.

The depegylation study results (not depicted in any Figure) showed no increase in depegylation over time in the samples stored at 4° C. and at 25° C. Even the samples stored at 40° C. only showed a 0.1% increase in depegylation, and only after 12 weeks of storage at that temperature.

We claim:

1. A method of making a modified antibody formulation, comprising:
a) providing a pre-lyophilized modified antibody solution comprising molecules capable of adversely affecting the stability or solubility of the modified antibody after lyophilization, and a modified antibody;
b) removing at least some of the molecules from the pre-lyophilized modified antibody solution; and
c) lyophilizing the solution from step (b), producing a lyophilized modified antibody formulation.

2. The method of claim 1, the modified antibody comprising an antibody fragment covalently attached to at least one nonproteinaceous polymer.

3. The method of claim 2, wherein the at least one nonproteinaceous polymer is at least one poly(ethyleneglycol) polymer.

4. The method of claim 3, wherein the at least one poly(ethyleneglycol) polymer is at least two methoxypoly(ethyleneglycol) polymers.

5. The method of claim 2, wherein the at least one nonproteinaceous polymer is covalently attached to the antibody through a linker.

6. The method of claim 5, wherein the linker comprises a succinimide moiety covalently attached to the antibody fragment through a cysteine residue of the antibody fragment.

7. The method of claim 6, wherein the linker further comprises a lysine residue that is covalently attached to the succinimide moiety and to the at least one nonproteinaceous polymer.

8. The method of claim 1, wherein the modified antibody is CDP870.

9. The method of claim 1, the molecules capable of adversely affecting the stability or solubility of the modified antibody after lyophilization provided in step (a) are smaller than the modified antibody.

10. The method of claim 1, wherein the molecules are removed in step (b) by dialysis.

11. The method of claim 1, wherein the molecules are removed in step (b) by diafiltration.

12. The method of claim 1, wherein at least 90% of the molecules are removed in step (b).

13. The method of claim 1, wherein the molecules removed in step (b) are salt molecules.

14. The method of claim 1, wherein the pre-lyophilized modified antibody solution provided in step (a) further comprises a volatile buffer, the method further comprising exchanging the volatile buffer for a non-volatile physiologically compatible buffer in step (b).

15. The method of claim 1, wherein the solution lyophilized in step (c) further comprises at least one excipient to facilitate reconstitution of the lyophilized modified antibody in a reconstitution solution.

16. The method of claim 15, wherein the at least one excipient is selected from the group consisting of a surfactant and a sugar.

17. The method of claim 1, further comprising a step of reconstituting the lyophilized modified antibody in a reconstitution solution, producing a formulation of reconstituted modified antibody.

18. The method of claim 17, wherein the formulation of reconstituted modified antibody has a modified antibody concentration of about 100 mg/ml to about 300 mg/ml.

19. The method of claim 17; wherein the formulation of reconstituted modified antibody has a modified antibody concentration of at least about 300 mg/ml to about 450 mg/ml.

20. An antibody formulation produced according to the method of claim 1.

21. A method of making a formulation of CDP870, comprising:
a) providing a pre-lyophilized solution comprising: CDP870 and molecules capable of adversely affecting the stability or solubility of CDP870 after lyophilization;
b) removing at least some of the molecules from the pre-lyophilized solution; and
c) lyophilizing the solution from step (b), producing a lyophilized CDP870 formulation.

22. The method of claim 21, wherein the molecules are removed in step (b) by dialysis.

23. The method of claim 1, wherein the molecules are removed in step (b) by diafiltration.

24. The method of claim 1, wherein at least 90% of the molecules are removed in step (b).

25. The method of claim 1, wherein the molecules removed in step (b) are salt molecules.

26. The method of claim 1, wherein the pre-lyophilized solution provided in step (a) further comprises a volatile buffer, the method further comprising exchanging the volatile buffer for a non-volatile physiologically compatible buffer in step (b).

27. The method of claim 1, wherein the solution lyophilized in step (c) further comprises at least one excipient to facilitate reconstitution of the lyophilized CDP870 formulation in a reconstitution solution.

28. The method of claim 27, wherein the at least one excipient is selected from the group consisting of a surfactant and a sugar.

29. The method of claim 1, further comprising a step of reconstituting the lyophilized CDP870 formulation in a reconstitution solution, producing a formulation of reconstituted CDP870.

30. The method of claim 29, wherein the formulation of reconstituted CDP870 has a concentration of about 100 mg/ml to about 300 mg/ml CDP870.

31. The method of claim 29, wherein the formulation of reconstituted CDP870 has a concentration of at least about 300 mg/ml to about 450 mg/ml CDP870.

32. A formulation of CDP870 produced according to the method of claim 21.

33. A method of treating or preventing a condition or disease in a mammalian subject, comprising:
a) providing a reconstituted lyophilized formulation of CDP870 produced by, prior to lyophilization, removing molecules capable of adversely affecting the stability or solubility of CDP870 after lyophilization; and
b) administering a pharmaceutically effective amount of the reconstituted lyophilized formulation of CDP870 to the subject.

34. The method of claim 33, wherein the molecules are removed prior to lyophilization by dialysis.

35. The method of claim 33, wherein the molecules are removed prior to dialysis by diafiltration.

36. The method of claim 33, wherein the subject is a human being.

37. The method of claim 33, wherein the disease treated or prevented according to the method is selected from the group consisting of: primary biliary cirrhosis; Myelodysplastic syndrome; chronic variable immunodefficientcy; treatment refractory sarcoidosis; diffuse lung disease, such as pulmonary fibrosis that is idiopathic or secondary to RA, or acute interstitial pneumonitis; vasculitis, such as Wegeners vasculitis, polyarteritis nodosa, temporal arteritis, IgA nephropathy (Henoch-Schonlein Purpura); crescentic nephritisjuvenile treatment resistant uveitis; adult treatment resistant uveitis; primary sclerosing cholangitis, alcohol induced hepatitis, ulcerative colitis, inflammatory skin diseases, such as bullous pemhigoid, and pemphigus vulgaris; polyositis (dermatomyositis); or an inflammatory disease, such as endotoxic shock associated with bacterial sepsis or a chronic disease such as rheumatoid arthritis, Crohn's disease, ulcerative colitis, and multiple sclerosis.

38. The method of claim 33, wherein the disease treated or prevented according to the method is rheumatoid arthritis.

39. A high concentration modified antibody formulation, comprising a modified antibody in a diluent for a modified antibody concentration of at least about 300 mg/ml.

40. The formulation of claim 39, the modified antibody comprising an antibody fragment covalently attached to at least one nonproteinaceous polymer.

41. The formulation of claim 40, wherein the at least one nonproteinaceous polymer is at least one poly(ethyleneglycol) polymer.

42. The formulation of claim 41, wherein the at least one poly(ethyleneglycol) polymer is at least two methoxypoly(ethyleneglycol) polymers.

43. The formulation of claim 40, wherein the at least one nonproteinaceous polymer is covalently attached to the antibody through a linker.

44. The formulation of claim 40, wherein the linker comprises a succinimide moiety covalently attached to the antibody fragment through a cysteine residue of the antibody fragment.

45. The formulation of claim 44, wherein the linker further comprises a lysine residue that is covalently attached to the succinimide moiety and to the at least one nonproteinaceous polymer.

46. The formulation of claim 45, wherein the modified antibody is CDP870.

47. The formulation of claim 39, wherein the concentration of modified antibody is about 300 mg/ml to about 450 mg/ml.

48. The formulation of claim 39, wherein the diluent is an aqueous solution.

49. The formulation of claim 48, wherein the diluent comprises a buffer that maintains the pH of the antibody formulation from about 4.5 to about 6.0.

50. The formulation of claim 39, wherein the high concentration modified antibody formulation has been produced by removing at least some molecules capable of adversely affecting the stability or solubility of the modified antibody after lyophilization from a pre-lyophilized modified antibody solution, lyophilizing the solution, and reconstituting the resulting lyophilized modified antibody in an appropriate volume of the diluent to produce the high concentration modified antibody formulation.

51. The formulation of claim 50, wherein the at least some molecules are removed by dialysis prior to lyophilizing.

52. The formulation of claim 50, wherein the at least some molecules are removed by diafiltration prior to lyophilizing.

53. The formulation of claim 39, wherein the high concentration formulation of modified antibody has been produced by concentrating a solution comprising a lower concentration of the modified antibody, by concentrating equilibrium dialysis.

54. A high concentration formulation of CDP870, comprising CDP870 in a diluent for a CDP870 concentration of at least about 300 mg/ml.

55. The formulation of claim 54, wherein the concentration of CDP870 is about 300 mg/ml to about 450 mg/ml.

56. The formulation of claim 54, wherein the diluent is an aqueous solution.

57. The formulation of claim 54, wherein the diluent comprises a buffer that maintains the pH of the antibody formulation from about 4.5 to about 6.0.

58. The formulation of claim 54, wherein the high concentration CDP870 formulation has been produced by removing at least some molecules capable of adversely affecting the stability or solubility of CDP870 after lyophilization from a pre-lyophilized modified antibody solution, lyophilizing the solution, and reconstituting the resulting lyophilized CDP870 in an appropriate volume of the diluent to produce the high concentration CDP870 formulation.

59. The formulation of claim 58, wherein the at least some molecules are removed by dialysis prior to lyophilizing.

60. The formulation of claim 58, wherein the at least some molecules are removed by diafiltration prior to lyophilizing.

61. The formulation of claim 54, wherein the high concentration formulation of CDP870 has been produced by concentrating a solution comprising a lower concentration of CDP870, by concentrating equilibrium dialysis.


SEQUENCE LISTING (2 sequences)

SEQ ID NO: 1 - protein, 208 aa, Artificial
  Grafted Light Chain for Fab and Modified Fab
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSGESVTEQDSKDSTYSLSSTLTLSKADYEKHKVYACEVTHQGLSSPVTKS

SEQ ID NO: 2 - protein, 229 aa, Artificial
  Grafted Heavy Chain for Modified Fab
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVAHKPSNTKVDKKVEPKSCDKTHTCAA